(19) United States
(12) Patent Application Publication
(10) Pub. No.: US 2025/0139692 A1
(43) Pub. Date: May 1, 2025

(54) DENTAL CARE AUCTION SYSTEM AND METHOD

(71) Applicant: Kamel A. Elhosary, Austin, TX (US)

(72) Inventor: Kamel A. Elhosary, Austin, TX (US)

(21) Appl. No.: 18/927,093

(22) Filed: Oct. 25, 2024

Related U.S. Application Data
(60) Provisional application No. 63/545,838, filed on Oct. 26, 2023.

(51) Int. Cl. G06Q 30/08; G16H 20/40

(52) U.S. Cl. CPC G06Q 30/08; G16H 20/40

(57) ABSTRACT

According to one example, a device includes one or more memory units, and one or more processors communicatively coupled to the one or more memory units. The processor(s) are configured to receive a request for a dental treatment for a dental patient, receive one or more bids for the dental treatment for the dental patient, and determine a winning bid for the dental treatment for the dental patient. The winning bid is associated with a dental care provider that will provide the dental treatment for the dental patient at a price included in the winning bid. There may be no human interaction between the dental patient and the dental care provider between a time when the request is received and a time when the winning bid is determined.

### CROSS REFERENCE TO RELATED APPLICATIONS

This application claims the benefit of priority to U.S. Provisional Patent Application No. 63/545,838, filed Oct. 26, 2023, the entirety of which is incorporated herein by reference.

### TECHNICAL FIELD

This disclosure relates generally to a healthcare, and more specifically to a dental care auction system and method that may operate with little to no human interaction.

### BACKGROUND

Dental care and the negotiation of prices for dental care traditionally requires human interaction. For example, a dental patient may go to a dental care provider (e.g., dentist), where they may be diagnosed with a dental issue (e.g., need a crown). The dentist may then quote a price to the dental patient for treating the dental issue. The dental patient can then accept the price, or not have the dental procedure performed. This traditional system and method for dental care and the negotiation of prices for dental care, however, may be deficient.

### SUMMARY

According to one example, a device includes one or more memory units, and one or more hardware processors communicatively coupled to the one or more memory units. The hardware processor(s) are configured to receive a request for a dental treatment for a dental patient; receive one or more bids for the dental treatment for the dental patient; and determine a winning bid for the dental treatment for the dental patient. The winning bid is associated with a dental care provider that will provide the dental treatment for the dental patient at a price included in the winning bid. There may be no human interaction between the dental patient and the dental care provider between a time when the request is received and a time when the winning bid is determined.

### BRIEF DESCRIPTION OF THE FIGURES

For a more complete understanding of the present disclosure and one or more examples of the features and advantages of the present disclosure, reference is now made to the following description, taken in conjunction with the accompanying drawings, in which:

FIG. 1 illustrates an example auction system 10.

FIG. 2 illustrates an example method of implementation of the auction system 10 of FIG. 1.

### DETAILED DESCRIPTION

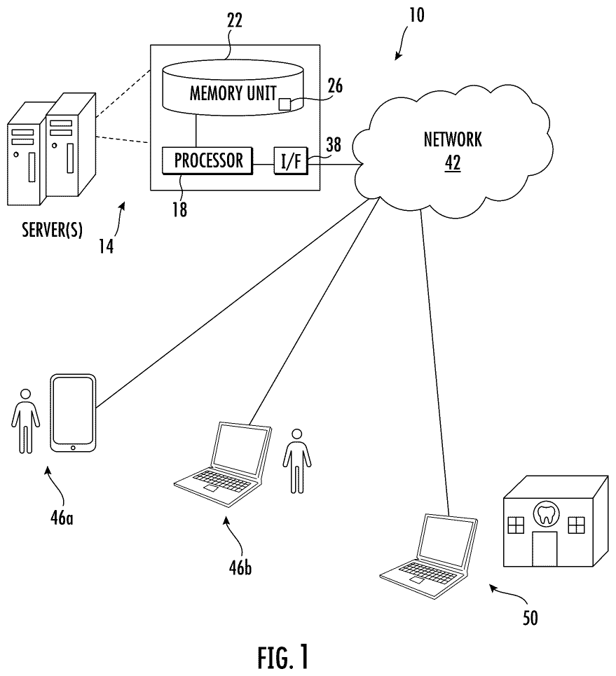

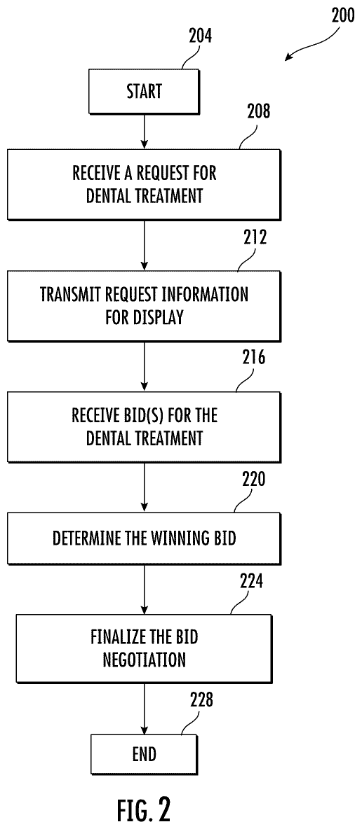

Examples in the present disclosure are best understood by referring to FIGS. 1-2 of the drawings, like numerals being used for like and corresponding parts of the various drawings.

Dental care is traditionally expensive. Furthermore, the high cost of dental procedures tends to be a large barrier to treatment. On one hand, patients would like to have their teeth fixed and yet cannot afford it (or do not want to pay for it), and on the other hand, dentists need to generate enough revenue to pay their bills and net a profit.

Dental care and the negotiation of prices for dental care traditionally requires human interaction. For example, a dental patient may go to a dental care provider (e.g., dentist), where they may be diagnosed with a dental issue (e.g., need a crown). The dentist may then quote a price to the dental patient for treating the dental issue. The dental patient can then accept the price, or not have the dental procedure performed.

The traditional systems and methods for dental care and the negotiation of prices for dental care, however, may be deficient. In particular, the high cost of dental procedures tends to dissuade a dental patient from undergoing various dental procedures that may be needed (e.g., a tooth filling due to a cavity). Furthermore, although a dental care provider needs to be able to pay their bills, in many cases, some money is better than no money at all. This is particularly true when the dental care provider has openings in their daily schedule that they cannot typically fill. To that end, the dental care provider may not mind offering a discount to get the wheel spinning.

Unfortunately, human interaction tends to get in the way of dental care price negotiation between the dental patient and the dental care provider. For example, if a dental care provider offers to perform a dental procedure for $1,500, it tends to be awkward for the dental care provider to then subsequently reduce that price if the patient turns it down. Furthermore, it may also make the dental care provider look like they were initially overcharging with their first price. The same can be said for the dental patient. If the dental patient cannot afford a needed $1,500 dental procedure, it may be awkward for the dental patient to try and explain that to the dental care provider and then ask for a discount or ask for the dental care provider's lowest price. Also, it tends to be difficult for a dental patient to get a second opinion with a lower price quote. For example, most dental care providers tend to not provide quotes over the telephone or without actually seeing the dental patient for a consultation. As such, an attempt to get a second opinion with a possible lower price quote tends to require the dental patient paying for another consultation (which can reduce the savings to the dental patient even if the quote is less). Finally, even a third party intermediary does not really fix this issue. While third party intermediaries can keep a dental patient and a dental care provider separate from each other, such third party intermediaries are also humans. As such, they can be susceptible to biases and favoritism, either of which can unfairly increase the price of dental treatment.

In contrast to this, in some examples, the dental care system 10 of FIG. 1 may address one or more of these deficiencies. For example, the dental care system 10 may include an auction system 14 that may operate with no human interaction (or a reduction in human interaction) between the dental patient and dental care provider(s). This may help reduce awkward money scenarios between the dental patient and dental care provider(s), which can reduce the cost of the dental procedure for the dental patient (allowing the dental patient to afford the treatment they need), and can also increase profits for dental care providers (e.g., by filling in open spots in a dental care provider's daily schedule).

FIG. 1 illustrates an example dental care auction system 10 that utilizes an auction device 14. In the example illustrated in FIG. 1, system 10 includes one or more auction devices 14, one or more networks 42, one or more patient devices 46, and one or more provider devices 50. Generally, the auction device 14 may allow a dental patient to submit their dental issue (using the patient device 46) to the auction device 14 over the network(s) 42, and the auction device 14 may allow a dental care provider (e.g., dentist) to submit open or closed bids (using the provider device 50) for treating the dental issue to the auction device 14 over the network(s) 42. Then, if the bid is accepted, the dental patient can receive (and the dental care provider can provide) the treatment for the dental issue at a price that may be acceptable to both the dental patient and the dental care provider. As is seen in the example illustrated in FIG. 1, the auction device 14 may be separated (e.g., at an arm's length) from both the dental patient and the dental care provider. As such, the auction device 14 may operate with no human interaction (or a reduction in human interaction) between the dental patient and dental care provider(s). No human interaction between the dental patient and dental care provider refers to a situation where the dental patient does not communicate (e.g., in-person conversation, via phone, via message(s)) or come in contact with the dental care provider (or vice versa) when negotiating the price of treating the dental issue. As such, the auction device 14 may prevent the awkward money-based situations between the dental patient and the dental care provider, in some examples.

In the example illustrated in FIG. 1, the system 10 includes the auction device 14. The auction device 14 may be any device that receives data, stores data, transmits data, and/or presents data for display to a user. For example, the auction device 14 may be (or may include) a personal computer, a laptop, a computer tablet, an iPad™, a mobile telephone (such as a Smartphone), an electronic notebook, a personal digital assistant, a host computer, a workstation, a web server, a network server, any suitable remote server, a mainframe, a file server, any other computing or processing device, any other device for receiving data, storing data, transmitting data, and/or presenting data for display to a user, or any combination of the preceding. The functions of the auction device 14 may be performed by any suitable combination of one or more servers or other components at one or more locations. In an example where the auction device 14 is a server, the server may be a private server, and the server may be a virtual or physical server. The server may include one or more servers at the same or remote locations. Also, the auction device 14 may be (or may include) any suitable component that functions as a server. In the example illustrated in FIG. 1, the auction device 14 is a server that hosts a website that may operate as an online store, marketplace, and/or auction site. Furthermore, in the example illustrated in FIG. 1, the auction device 14 includes a computer/data processor 18 (e.g., hardware processor) and data storage medium 22 (e.g., memory unit) comprising instructions 26 executable by the processor 18 to perform the functions of the auction device 14, a display (not shown) that may present data for display to a user (e.g., to an administrator of the auction device 14), and a communication port 38 (e.g., receiver, transmitter, transceiver, network interface) configured to receive and transmit data (e.g., receive requests for data, transmit data). Further example details of auction device 14 and/or its components are discussed below.

In the example illustrated in FIG. 1, the system 10 also includes one or more networks 42. The network 42 represents any suitable network operable to facilitate communication between the components of system 10, such as auction devices 14, patient devices 46, and provider devices 50. The network 42 may include any interconnecting system capable of transmitting audio, video, signals, data, information, messages, or any combination of the preceding. The network 42 may include all or a portion of a public switched telephone network (PSTN), a public or private data network, a local area network (LAN), a metropolitan area network (MAN), a wide area network (WAN), a local, regional, or global communication or computer network, such as the Internet, a wireline or wireless network, an enterprise intranet, any other suitable communication link, including combinations thereof, operable to facilitate communication between the components of system 10, or any combination of the preceding.

In the example illustrated in FIG. 1, the system 10 further includes one or more patient devices 46. The patient device 46 may be any device that receives data, stores data, transmits data, and/or presents data for display. For example, the patient device 46 may be (or may include) a personal computer, a laptop, a mobile telephone (such as a Smartphone), an electronic notebook, a personal digital assistant, a host computer, a workstation, any other computing or processing device, any other device for receiving data, storing data, transmitting data, and/or presenting data for display to a user, or any combination of the preceding. In the example illustrated in FIG. 1, the patient device 46a is a Smartphone and the patient device 46b is a laptop. In the example illustrated in FIG. 1, each patient device 46 is associated with a particular dental patient. The dental patient (or an associate of the dental patient, such as the dental patient's parent, sibling, or any other person authorized to act on the dental patient's behalf) may use the patient device 46 to interact with the auction device 14 in order to negotiate the cost of treating a dental issue.

In the example illustrated in FIG. 1, the system 10 further includes one or more provider devices 50. The provider device 50 may be any device that receives data, stores data, transmits data, and/or presents data for display. For example, the provider device 50 may be (or may include) a personal computer, a laptop, a mobile telephone (such as a Smartphone), an electronic notebook, a personal digital assistant, a host computer, a workstation, any other computing or processing device, any other device for receiving data, storing data, transmitting data, and/or presenting data for display to a user, or any combination of the preceding. In the example illustrated in FIG. 1, the provider device 50 is a laptop. In the example illustrated in FIG. 1, each provider device 50 is associated with a particular dental care provider (e.g., dentist, dental office). The dental care provider (or an associate of the dental care provider, such as any person authorized to act on the dental care provider's behalf) may use the provider device 50 to interact with the auction device 14 in order to negotiate the cost of treating a dental issue.

Modifications, additions, or omissions may be made to system 10 without departing from the scope of the disclosure. For example, system 10 may include any number of auction devices 14, networks 42, patient devices 46, and/or provider device 50 (and/or any number of components, such as processors or memory units illustrated or described in the above devices). Also, any suitable logic may perform the functions of system 10 and the components and/or devices within system 10. Furthermore, one or more components of system 10 may be separated, combined, and/or eliminated.

FIG. 2 illustrates an example method 200 of implementation of the auction system 10 of FIG. 1. In the illustrated example, each of the steps of method 200 are performed by the auction device 14 (e.g., performed by the processor(s) 18 of the auction device 14). In other examples, one or more of the steps of method 200 may be performed by any other device (e.g., any other device of FIG. 1) or entity. Additionally, for ease of explanation and understanding but not by way of limitation, method 200 is described below with regard to dental care (e.g., dentistry, orthodontia, pediatric dentistry). However, this disclosure contemplates the use of the auction system 10 in any healthcare industry or area, in some examples.

The method 200 starts at step 204. At step 208, a request for dental treatment is received. The request for dental treatment may be received from any person, and for any reason. For example, the request for dental treatment may be received from dental patient who has previously been diagnosed with a particular dental issue. In such an example, the dental patient may have gone to a first dental care provider (e.g., due to pain, for a regular check-up), and received a diagnosis for a dental issue (e.g., need a crown). However, in this example, the dental patient has refused to have the dental issue treated by the first dental care provider. In some examples, this refusal may be due to the cost being too high. For example, the first dental care provider may have quoted $1,500 to provide the dental patient with a crown, and the dental patient refused the procedure because the dental patient is only willing (or only able) to pay, for example, $1,000 for the crown. Accordingly, in the example discussed above, the dental patient may utilize a patient device 46 (e.g., a Smartphone) to submit a request for dental treatment to the auction system 14.

The request for dental treatment may be received in any manner. For example, the auction device 14 may host a website that allows the dental patient to utilize a patient device 46 (e.g., a Smartphone) to submit a request for dental treatment to the auction system 14. As another example, the auction device 14 may be a web application server that the dental patient can communicate with using a web browser on the patient device 46 or a web application installed on the patient device 46.

To submit the request, the dental patient may first register with the auction device 14 (e.g., by providing their name, address, password, etc.). Once registered, the dental patient may submit the request for dental treatment (e.g., via a website, web application interface), and this request may be received by the auction device 14.

The request may include any information that may be used to assess the request for dental treatment. Examples of such information include the dental issue (e.g., need a crown), images and/or other data associated with the dental issue (e.g., diagnosis notes, x-ray images), preferred deadline for treatment (e.g., a particular date, a timeframe, etc.), preferred location for treatment (e.g., within a 25 mile radius of the dental patient's address), insurance information, any other information, or any combination of the preceding.

In some examples, the request may include a closed bid auction request. A closed bid auction request refers to a request where the dental patient provides a maximum cost that the dental patient is willing to pay for the treatment. This maximum cost may be displayed to the dental care providers, or it may not be displayed to the dental care providers. If a dental care provider submits a bid that is less than or equal to the maximum cost, the dental care provider is automatically selected as the winner of the closed bid auction, in some examples.

In some examples, the request may include an open bid auction request. An open bid auction request refers to a request where the dental patient does not provide a maximum cost that the dental patient is willing to pay for the treatment. In such an example, each dental care provider may submit a bid for what price they are willing to charge to treat the dental patient. In this example, the lowest bid (at the end of a particular predetermined time) is automatically selected as the winner of the open bid auction, in some examples. In other examples, the dental patient may be allowed to select which dental care provider is the winner of the open bid auction (even if the bid was not the lowest). As one example of this, a dental patient may be allowed to select a higher rated dental care provider as the winner, even if their bid was not the lowest. In some examples, the dental patient be allowed to select any of the bids (even if the bid was not the lowest). In other examples, the dental patient may only be allowed to select bids that are within a particular percentage (e.g., within 5 percent) or amount (e.g., within $200) of the lowest bid.

In some examples, the request may include a hybrid bid auction request. A hybrid bid auction request refers to a request where the dental patient does provide a maximum cost that the dental patient is willing to pay for the treatment, but each dental care provider may also submit a bid for what price they are willing to charge to treat the dental patient. The lowest bid (at the end of a particular predetermined time) that is less than or equal to the maximum cost is automatically selected as the winner of the hybrid bid auction, in some examples. In other examples, the dental patient may be allowed to select which dental care provider is the winner of the hybrid bid auction (even if the bid was not the lowest). As one example of this, a dental patient may be allowed to select a higher rated dental care provider as the winner, even if their bid was not the lowest. In some examples, the dental patient be allowed to select any of the bids (even if the bid was not the lowest). In other examples, the dental patient may only be allowed to select bids that are within a particular percentage (e.g., within 5 percent) or amount (e.g., within $200) of the lowest bid.

As is discussed above, some of the auction types may result in an automatic selection of a dental care provider as the winner of the auction. In other examples, the dental care provider may need to meet certain other requirements (other than the bid) to win the auction. For example, the dental care provider also may need to satisfy the preferred deadline for treatment, may need to satisfy the preferred location for treatment, may need to accept particular payment types (e.g., credit card, with or without a surcharge), may need to accept particular insurance, may need to satisfy any other requirement, or any combination of the preceding. If the dental care provider cannot (or will not) meet the other requirements, the dental patient may have the option to refuse (or accept) an otherwise winning bid. In other examples, the dental care provider may not be allowed to even submit a bid unless the dental care provider also meets the other requirements. In some examples, certain dental care providers may not be allowed to be selected as a winner of an auction for a particular dental patient. For example, a dental patient may have had a bad experience with a particular dental care provider before. In such an example, the dental patient may indicate this in their request, or in their registration profile. As a result of this, that particular dental care provider may never be automatically selected as a winner of an auction. Instead, the dental patient may always be given the opportunity to refuse (or accept) a winning bid from that particular dental care provider.

In some examples, to assist a dental patient in submitting their request, the auction device 14 may provide particular information to the dental patient (e.g., before the dental patient submits their request). For example, the auction device 14 may include a look-up table (or scroll function) that will allow the dental patient to find (and/or select) their dental issue (even if they do not remember the official name of the treatment, or cannot spell it). As another example, the auction device 14 may include information about selected dental issues, such as details about the issue, typical treatment times, typical recovery times, cost ranges, any other information, or any combination of the preceding. This may assist the dental patient in preparing the request. For example, cost ranges for a treatment may allow the dental patient to more properly select a maximum cost.

Following step 208, the method 200 may move to step 212, where information associated with the request is transmitted for display. In this step, information associated with the request may be transmitted to the dental care providers, so as to allow the dental care providers to assess the dental care request, and submit (or not submit) a bid. In some examples, the information is transmitted to the provider devices 50 of FIG. 1. The information may be transmitted to the provider devices 50 in any manner. For example, the information may be displayed on the website accessed by the provider device 50, transmitted for view on a web browser on the provider device 50, transmitted for view on a web application installed on the provider device 50, transmitted as an e-mail to an account accessible on the provider device 50, transmitted as a text message to the provider device 50, any other manner of transmission, or any combination of the preceding.

The information transmitted may be any information that can be used to assess the request. For example, the information transmitted may include non-confidential patient information, the dental issue (e.g., need a crown), images and/or other data associated with the dental issue (e.g., diagnosis notes, x-ray images), preferred deadline for treatment (e.g., a particular date, a timeframe, etc.), preferred location for treatment (e.g., within a 25 mile radius of the dental patient's address), auction type (e.g., closed, open, hybrid), maximum cost (if applicable and/or if allowed by the dental patient) any other information, or any combination of the preceding.

The information may be transmitted to any number of dental care providers. For example, the information may be transmitted to all of the dental care providers that are registered with the auction device 14, all of the dental care providers that are registered and that meet certain requirements (e.g., location requirements, degree requirements, etc.), or any other number of dental care providers or subset of dental care providers.

Following step 212, the method 200 may move to step 216 where one or more bids for the dental treatment are received. The bid for the dental treatment may be received in any manner. For example, as is discussed above, the auction device 14 may host a website that allows the dental care provider to utilize the provider device 50 (e.g., a laptop) to submit a bid for the dental treatment to the auction device 14. As another example, the auction device 14 may be a web application server that the dental care provider can communicate with using a web browser on the provider device 50 or a web application installed on the provider device 50.

To submit the request, the dental care provider may first register with the auction device 14 (e.g., by providing their name, address, password, etc.). Once registered, the dental care provider may access information associated with each request for dental treatment (see step 212), and may further submit bids for the dental treatment (e.g., via a website, web application interface). These bids may then be received by auction device 14.

The bid may include any information associated with the dental treatment and/or the dental care provider. Examples of such information include the bid by the particular dental care provider (e.g., the price for the treatment), a minimum possible bid (e.g., the bid provided by the particular dental care provider may automatically be lowered by increments to the minimum possible bid, if another dental care provider has submitted a lower bid), treatment date (e.g., a particular date, a timeframe, etc.), location for treatment (e.g., address of the dental care provider), any other information, or any combination of the preceding. The bid may also include information that indicates whether the dental care provider agrees to certain additional requirements submitted by the dental patient (e.g., accepts credit card without a surcharge, accepts insurance).

In some examples, the auction device 14 may restrict certain bid amounts. For example, each dental care provider may have a profile with the auction device 14. This profile may include information on typical charges from that dental care provider for particular dental treatments. If the bid submitted by a dental care provider is more than those typical charges (e.g., by a particular percentage or amount), the bid may be denied.

Any number of bids may be received at step 216, and bids may be received from any number of dental care providers. In some examples, a dental care provider may only be allowed to provide a single bid for each request. In other examples, a dental care provider may provide multiple bids for each request.

Following step 216, the method 200 may move to step 220 where the winning bid is determined. The winning bid may be determined in any manner. For example, the auction device 14 may determine the winning bid by automatically selecting a winning bid (e.g., if it is the lowest). In another example, the auction device 14 may determine the winning bid by receiving a selection of a bid from the dental patient (via the patient device 46).

Following step 220, the method 200 may move to step 224, where the bid/price negotiation is finalized. This step 220 may include any number of additional steps that allow the bid negotiation to be finalized. For example, the step may include a transmittal of dental treatment information to the dental care patient and/or the dental care provider. Such information may include a confirmation of the winning bid and confirmation of pending dental treatment, confirmation of treatment date (e.g., a particular date, a timeframe, etc.), confirmation of location for treatment (e.g., address of the dental care provider), any other information, or any combination of the preceding. As another example, the step may include the request for and payment of a deposit by the dental patient for the dental treatment. In such an example, a percentage of the treatment cost may be required as a deposit, and this amount may be transmitted to the auction device 14 (and/or the dental care provider). In some examples, the deposit amount may be used to compensate the dental care provider if the dental patient does not show at the treatment date/location.

Following step 224, the method 200 moves to step 228, where the method ends. Modifications, additions, or omissions may be made to method 200. For example, one or more steps of method 200 may be optional, or may not be performed. As a further example, the steps of method 200 may be performed in parallel or in any suitable order.

In some examples, the method 200 may include further steps, and/or the system 10 may include additional capabilities. For example, in addition to receiving bids from dental care providers, the auction device 14 may also be able to receive advertisements, promotions, and/or discounts from the dental care providers. These advertisements, promotions, and/or discounts may be displayed to the dental patients when they access the auction device 14 (e.g., by logging in, accessing the website). In some examples, these advertisements, promotions, and/or discounts may include treatment costs for one or more dental procedures. As such, instead submitting a request for dental treatment, the dental patient may automatically choose one of the advertised costs.

In some examples, the dental care providers may offer office waitlists that include discounted treatment prices (e.g., for patients with financial difficulties). These waitlists may be submitted to the auction device 14, which may then be displayed to the dental patients. The dental patient may accept the waitlist price, in some examples, which may provide the dental patient with a treatment time frame (e.g., in the month of January) with a caveat that the dental patient will be informed of a firm date, time, and location of the treatment at a predetermined amount of time (e.g., 3 days) in advance. The treatment time frame may allow the dentist freedom to fill in schedule gaps closer to the actual date, but may require a lower cost for the treatment in order to do so, in some examples. In other examples, the waitlist may be for future promotions or discounts. If the dental patient signs up for the waitlist, the dental patient may receive promotions or discounts in the future. In some examples, the waitlists may be available for all dental patients. In other examples, the waitlists may only be available for current or preexisting patients of the dental care provider.

In various examples, the herein described systems (e.g., system 10), devices (e.g., auction device(s) 14, patient device(s) 46, provider device(s) 50), components of the devices, and methods may be implemented in software, firmware, or executable instructions stored in a data storage medium such as or including machine-readable medium. The term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure. Some examples may be implemented using a machine-readable medium or article which may store an instruction or a set of instructions that, if executed by a machine, may cause the machine to perform a method and/or operations in accordance with the examples. Such a machine may include, for example, any suitable processing platform, computing platform, computing device, processing device, computing system, processing system, computer, processor, or the like, and may be implemented using any suitable combination of hardware and/or software. Computers and computer systems described herein (e.g., auction device(s) 14, patient device(s) 46, provider device(s) 50) may include operatively associated computer-readable memory media such as memory for storing software applications and instructions used in obtaining, processing, storing or communicating data. It can be appreciated that such memory can be internal, external, remote or local with respect to its operatively associated computer or computer system. Memory may also include any manner of storing software or other instructions including, for example and without limitation, solid state RAM storage, a hard disk, an optical disk, floppy disk, DVD, compact disc, memory stick, flash drive, compact disc flash drive, ROM (read only memory), RAM (random access memory), PROM (programmable ROM), EEPROM (extended erasable PROM), or other like computer-readable media. The machine-readable medium or article may include, for example, any suitable type of memory unit, memory device, memory article, memory medium, storage device, storage article, storage medium and/or storage unit, for example, memory, removable or non-removable media, solid state random access media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, various types of Digital Versatile Disk (DVD), a tape, a cassette, or the like.

It will be further apparent to one of ordinary skill in the art that some of the examples as described hereinabove may be implemented in many different examples of instruction (e.g., software or firmware) and hardware. The instructions may include any suitable type of code, such as source code, compiled code, interpreted code, executable code, static code, dynamic code, and the like. The actual software code or specialized control hardware used to implement some of the illustrated examples do not limit the present disclosure. The instructions may be implemented, for example, using any suitable programing language, which may include high-level, low-level, object-oriented, visual, compiled or interpreted programming languages, such as, but not limited to, C, C++, C #, Java, BASIC, SQL, Perl, Matlab, Pascal, Visual BASIC, Go, Python, R, Java Script, Typescript, Objective C, Swift, assembly language, machine code, and so forth. The examples are not limited in this context.

The methods (e.g., method 200), systems (e.g., system 10), devices (e.g., auction device(s) 14, patient device(s) 46, provider device(s) 50), and components of the devices have been illustrated and described herein as comprising several separate functional elements, such as modules or units. Although certain of such modules or units may be described by way of example, it can be appreciated that a greater or lesser number of modules or units may be used and still fall within the scope of the examples. Further, although various examples may be described in terms of modules or units to facilitate description, such modules or units may be implemented by one or more hardware components (e.g., embedded systems/peripherals, processors, chips, FPGAs, DSPs, PLDs, ASICs, circuits, registers, servers, clients, network switches and routers), software components (e.g., programs, subroutines, logic) and/or combination thereof. It can be appreciated that, in certain aspects, a single component may be replaced by multiple components, and multiple components may be replaced by a single component, to provide an element or structure or to perform a given function or functions. Except where such substitution would not be operative to practice certain examples of the present disclosure, such substitution is considered within the scope of the present disclosure. In one example, all or a portion of the system, its features or functional elements, modules, units, etc. or one or more steps of the method may be associated with, implemented by, executed on, or embedded in (e.g., as embedded software/firmware) one or more hardware components. Further, such one or more components so configured may be installed or associated with one or more devices and therein configured to perform the herein described system functionalities or methods. The modules or units may comprise, or be implemented as, one or more systems, sub-systems, devices, components, circuits, logic, programs, or any combination thereof, as desired for a given set of design or performance constraints. For example, the modules may comprise electronic elements fabricated on a substrate. In various implementations, the electronic elements may be fabricated using silicon-based IC processes such as complementary metal oxide semiconductor (CMOS), bipolar, and bipolar CMOS (BiCMOS) processes, for example. The examples are not limited in this context.

It may be appreciated that terms such as “processing”, “generating”, “determining”, or the like, unless stated otherwise, refer to the action or processes of a computer or computing system, or similar electronic computing device, that manipulates or transforms data represented as physical quantities (e.g., electronic) within the computing system's registers or memories into other data similarly represented as physical quantities within the computing system's memories, registers or other such information storage, transmission or display devices. The examples are not limited in this context. An action such as “identifying” or “matching” when performed by a computer or computer system may include identification by determining, accessing system data, comparisons with system data, instructions, or the like. An action such as initiating may include causing an event or thing initiated either directly or indirectly. For example, initiating may include signaling, providing power or instructions, physical manipulation, transmission of data, calculation of conditions, or other step resulting in the event sought to be initiated. Furthermore, an action such as “storing”, when used in reference to a computer or computer system, refers to any suitable type of storing operation including, for example, storing a value to memory, storing a value to cache memory, storing a value to a processor register, and/or storing a value to a non-volatile data storage device.

This specification has been written with reference to various non-limiting and non-exhaustive examples. However, it will be recognized by persons having ordinary skill in the art that various substitutions, modifications, or combinations of any of the disclosed examples (or portions thereof) may be made within the scope of this specification. Thus, it is contemplated and understood that this specification supports additional examples not expressly set forth in this specification. Such examples may be obtained, for example, by combining, modifying, or reorganizing any of the disclosed components, elements, features, aspects, characteristics, limitations, and the like, of the various non-limiting and non-exhaustive examples described in this specification. In this manner, Applicant reserves the right to amend the claims during prosecution to add features as variously described in this specification.

What is claimed is:

1. A device comprising:
one or more memory units;
one or more hardware processors communicatively coupled to the one or more memory units, the one or more hardware processors configured to:
receive a request for a dental treatment for a dental patient;
receive one or more bids for the dental treatment for the dental patient; and
determine a winning bid for the dental treatment for the dental patient, wherein the winning bid is associated with a dental care provider that will provide the dental treatment for the dental patient at a price included in the winning bid, wherein there is no human interaction between the dental patient and the dental care provider between a time when the request is received and a time when the winning bid is determined.

2. The device of claim 1, wherein the request for the dental treatment comprises one or more diagnosis notes associated with the dental patient and one or more x-ray images associated with the dental patient.

3. The device of claim 1, wherein the winning bid is not a bid of the one or more bids with the lowest price.

4. The device of claim 1, wherein the one or more hardware processors are further configured to:
determine that a first bid of the one or more bids is priced higher than a typical price for the dental treatment associated with the respective dental care provider that submitted the first bid; and
following the determination, reject the first bid.

5. A device comprising:
one or more memory units;
one or more processors communicatively coupled to the one or more memory units, the one or more processors configured to:
receive a request for a dental treatment for a dental patient;
receive one or more bids for the dental treatment for the dental patient; and
determine a winning bid for the dental treatment for the dental patient, wherein the winning bid is associated with a dental care provider that will provide the dental treatment for the dental patient at a price included in the winning bid.

6. A method comprising:
receiving, by a computing system, a request for a dental treatment for a dental patient;
receiving, by the computing system, one or more bids for the dental treatment for the dental patient; and
determining, by the computing system, a winning bid for the dental treatment for the dental patient, wherein the winning bid is associated with a dental care provider that will provide the dental treatment for the dental patient at a price included in the winning bid, wherein there is no human interaction between the dental patient and the dental care provider between a time when the request is received and a time when the winning bid is determined.
